(19) United States
(12) Patent Application Publication
(10) Pub. No.: US 2010/0203562 A1
(43) Pub. Date: Aug. 12, 2010

(54) PLASMA FILTRATION

(76) Inventors: Bernd Binder, Vienna (AT); Helga Vetr, Moedling (AT)

Correspondence Address: KF ROSS PC, 5683 RIVERDALE AVENUE, SUITE 203 BOX 900, BRONX, NY, 10471-0900, US

(21) Appl. No.: 12/667,266

(22) PCT Filed: Jul. 4, 2008
(86) PCT No.: PCT/AT2008/000245

§ 371 Date: Mar. 12, 2010

(30) Foreign Application Priority Data
Jul. 6, 2007 (AT) A 1045/2007

(51) Int. Cl. C12Q 1/56; C12M 1/34

(52) U.S. Cl. 435/13; 435/287.1

(57) ABSTRACT

The invention relates to a process for preparing blood plasma for determining one or more blood parameters, in particular coagulation parameters. In order to improve the measurement, the blood plasma is sucked through a filter by means of reduced pressure.

The invention relates to a method for the treatment of blood plasma before the determination of one or more blood parameters, such as for example coagulation parameters and thrombin parameters, the invention also relates to an apparatus that can carry out the method.

Unrelated EP 1 733 748 [US 2008/0058694] describes a method in which lipids are removed from blood plasma. The lipid-free blood plasma is then fed back to the blood. A determination of blood parameters is not provided.

Also unrelated EP 0 578 086 [U.S. Pat. No. 5,632,906] discloses a method in which a vacuum is used only to suction in or to transport the blood plasma as a whole. Filtering is not done.

To carry out various analyses, first plasma is obtained from the blood of a patient. This is carried out in most cases by centrifuging. In the prior art, the plasma obtained thereby forms the basis for various laboratory tests. The disadvantage of this method is that microparticles present in the blood are not selected by centrifuging, but remain in the plasma. It has now been proven that these microparticles falsify the measured values in the determination of diverse blood parameters.

There is therefore a need for a solution to this problem to achieve a reliable and reproducible determination of blood counts that are not falsified by the microparticles.

This object is attained with a method of the above-described type in that plasma obtained from the blood is filtered before it is analyzed for a specific parameter.

This object is attained with an apparatus of the above-described type in that a filter is provided through which the plasma reaches a collecting vessel, and a vacuum pump generates a vacuum on one side of the filter.

In a preferred embodiments the filter is essentially impermeable to microparticles, the filter has a transmission limit between 0.05 and 1.5 μm (micrometers), preferably between 0.1 and 1.2 μm (micrometers), the vacuum is −50 mbar to −1000 mbar, preferably −300 mbar to −600 mbar, and the impingement of the filter with a vacuum lasts between 30 seconds and 30 minutes.

The apparatus for the treatment of blood plasma for the determination of one or more blood parameters, in particular coagulation parameters, is characterized in that the apparatus has a filter, a collecting vessel downstream of the filter, and a vacuum pump connected to the region downstream of the filter.

In a preferred embodiment the filter has a transmission limit between 0.05 and 1.5 μm (micrometers), preferably between 0.1 and 1.2 μm (micrometers), that the collecting vessel is a microtitration plate, the collecting vessels are Eppendorf tubes, that the filter, the collection vessel and the vacuum pump are integrated into a common unit, and a cooler, preferably a Peltier element, is provided under the filter to cool the filtering operation.

The invention is explained in more detail based on the drawing. Therein:

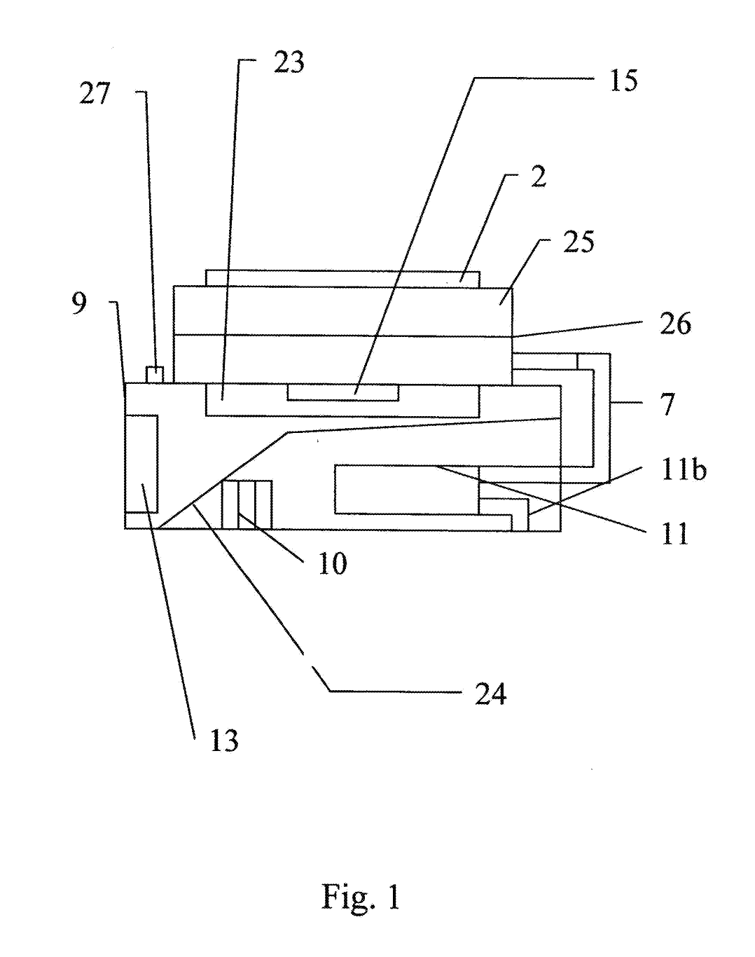

FIG. 1 is a diagrammatic illustration of an apparatus according to the invention,

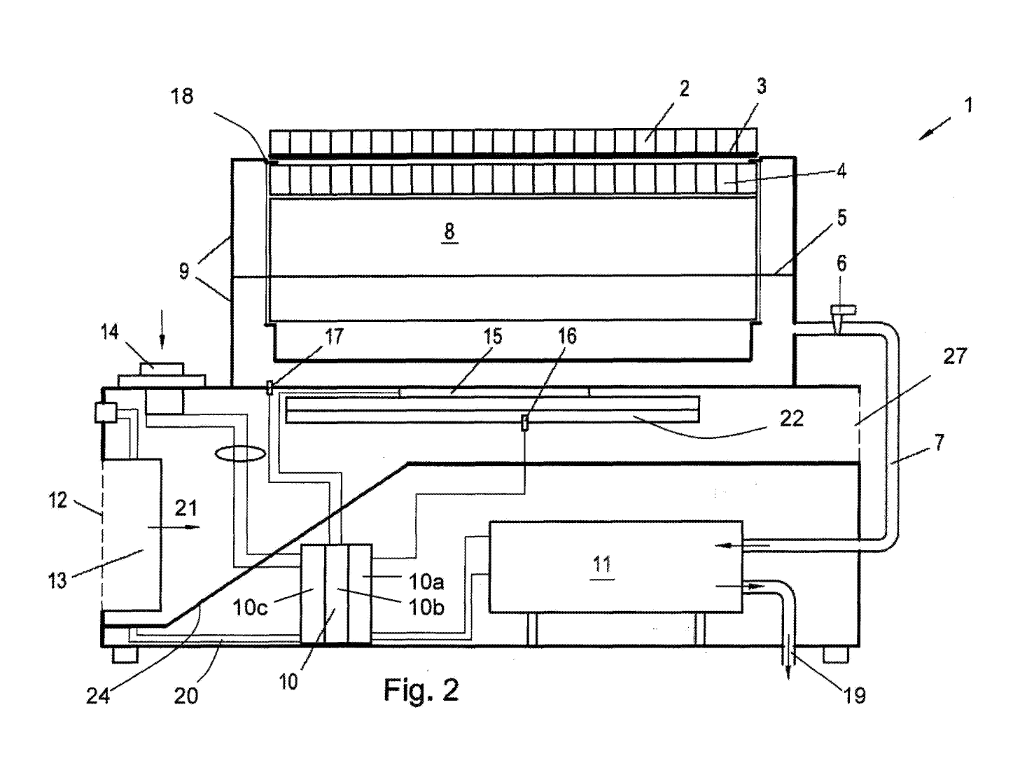

FIG. 2 is a detail view of an apparatus according to the invention.

Obtaining plasma from anticoagulated (citrate 0.011 mM) whole blood is usually carried out by centrifuging according to DIN [German Institute Standardization] 58905 (15 min., with at least 2500 g). The plasma obtained this way is described as platelet-poor plasma (PPP) and used to carry out various laboratory tests, in particular coagulation analyses. However, different quantities of microparticles (membranous vesicles of cells, 0.1 to 1 μm in size, in a number of ˜4,000 to ˜60,000/μl) are contained in this PPP and influence various coagulation tests, in particular thrombin generation. It is therefore desirable to use plasma that is free of microparticles for such laboratory analyses.

Although microparticles can be removed from the plasma by ultracentrifuging the plasma (100,000×g for 60 min¹), this procedure is time-consuming and also compromised in that one cannot prevent that as the microparticle-free plasma is taken up different quantities of microparticles are taken up too. Furthermore, microparticles are described that have the same density as plasma and therefore cannot be separated by centrifuging².

Although filtration units are described that can be used at best for the separation of plasma and microparticles via which a larger number of samples can be filtered via filter plates in matching collecting plates, these filtration units are connected to the membrane vacuum pumps customary in laboratories for this purpose. A relatively large amount of space is necessary for the hose lines, safety bottles, the pump itself and stop cocks necessary for this. The parameters used for the filtration are not standardized and remain up to the user.

The object of the present invention is to design a simple apparatus that permits rapid and standardized separation of plasma and microparticles.

In the apparatus (FIG. 1) on which the invention is based, a filtration unit comprising a membrane filter that is fitted on a collecting vessel in an air-tight manner is connected to a vacuum pump such that a vacuum is produced in the collecting vessel, and the vacuum sucks the plasma through the membrane filter and to obtain filtrate in the collecting vessel. The duration of the filtration is set by a controller connected to the vacuum pump and filtration pressure is set by adjustment of a valve. To control the filtration pressure, a pressure gauge is installed in the system. This results in a compact design requiring only a little space and makes possible a standardization of the filtration by setting the time and pressure. The vacuum pump is resistant to liquids and if necessary can optionally be cleaned together with the hose system with a wash solution. The filter assembly and the collecting plate for the samples can be cooled if necessary by a Peltier element in the base of the filter assembly.

The filtration unit comprises a housing with side covers on which commercially available filter assemblies, for example from Pall, can be mounted. A power supply furnishes power to the pump and other components.

The housing contains the following components: a switch to start the filtration operation and a socket for the power supply, a vacuum pump that produces the vacuum for the filtration, vacuum hoses from the filter assembly to the pump and from the pump to the outlet opening, and an electronic component for controlling the vacuum pump. In a preferred embodiment a Peltier element is provided for cooling the filter assembly and controlled by an electronic component. Furthermore, an axial-flow fan can be installed in the housing wall for cooling the heat sink, as well as an electronic component for controlling the axial-flow fan.

Process description of a filtration:

The filter assembly is equipped with a collecting plate and a filter plate. The samples are transferred with a pipette to the filter plate, the start button is pressed and the filtration starts automatically. At the end of the filtration, a pressure equalizer button is actuated so the filter plate holder can be lifted and the collecting plate with the samples can be removed.

If the filter assembly, the hoses, or the pump should get fouled, the dirt can be suctioned off with a washing solution from the filter installation to the outlet opening while the pump is running.

FIG. 1 shows in a diagrammatic form a plasma filtration apparatus according to the invention, whose he elements are integrated into a common unit. The filtration unit comprising an upper part 25 and a lower part 26 is located in the upper part of a housing 9. A Peltier element 15 under the filtration unit in interaction with a cooler 23, for example conductive metal, ensures an appropriate temperature of the plasma.

The filtration can be easily started with a start button 27 on the outside of the housing 9. The lower part of the apparatus according to the invention contains a vacuum pump 11 and a fan 13 for cooling a cooler 23 and all of the other electrically driven components. A corresponding baffle plate 24 is also provided for this purpose.

FIG. 2 shows in a somewhat more detailed manner, integrated into a common housing, an apparatus according to the invention for filtering blood plasma with a filter, a collecting vessel under the filter, a spacer block, and an opening via which the region below the filter can be acted on with a vacuum.

A vacuum pump is accommodated in the lower part of the unit and is connected to a controller. A cooler, preferably a Peltier element, is located in the upper region of the lower part of the unit and is thermally connected to cooling fins and to the controller for adjusting a specific temperature for the plasma to be filtered. An air intake on the left side with a fan ensures that heat generated by the Peltier element can be blown away. The parameters for the method can be adjusted via an input device, for example a keyboard.

FIG. 2 shows integrated into a common housing, for example an aluminum housing 9, an apparatus 1 according to the invention for filtering blood plasma in detail. On the top a multiple-well filter plate 2 is provided that is attached to the housing 9 with a full perimeter seal. The filter 3 is located on the underside of the multiple-well filter plate 2. A collecting vessel 4 is mounted below the filter, followed by a spacer block 8 and an opening via which the region below the filter can be acted on with a vacuum. A vacuum hose 7 provided with a stop cock 6 is used for this purpose, which vacuum hose connects the suction intake 11a of the vacuum pump 11 to the region below the filter. The outlet 11b of the vacuum pump 11 is connected to the air outlet 19 of the apparatus 1. A seal 5 is provided between the upper part and the lower part of the filter assembly.

A controller 10 comprises a temperature switch 10a for an axial-flow fan, a temperature switch 10b for a Peltier element and a long-term timer 10c. A power supply 20, for example furnishing 12V DC voltage, provides the controller 10 and the axial-flow fan 13 is with power. The axial-flow fan suctions air in via an air intake 12 and thus supplies the apparatus with cooling air 21. An input device 14 for controlling the apparatus 1 is provided on the outside of the housing 9.

A Peltier element 15 is provided under the filter assembly and has cooling fins 22 on its underside that are cooled by the air 21. A temperature sensor 16 is thermally connected to the cooling fins. A further temperature sensor 17 measures the temperature in the region above the Peltier element. The Peltier element and the temperature sensors 16 and 17 are connected to the controller 10, the transmission of drive pulses to the power supply being thus ensured.

A vacuum pump is accommodated in the lower part of the unit and is connected to a controller. A cooler, preferably a Peltier element, is in the upper region of the lower part of the unit and is thermally connected to cooling fins and to the controller for setting a specific temperature for the plasma to be filtered. An air intake on the left side with a fan ensures that heat generated by the Peltier element can be blown away. The parameters for the method can be adjusted via an input device, for example a keyboard.

### REFERENCE NUMBERS FOR FIGS. 1 AND 2

Apparatus 1,

Multiple-well filter plate, 2

Filter 3,

Collecting vessel 4,

Seal between upper part and lower part 5,

Stop cock 6,

Vacuum hose 7,

Spacer block 8,

Housing, for example aluminum housing 9,

Controller 10,

Temperature switch 10a for an axial-flow fan,

Temperature switch 10b for a Peltier element,

Long-term timer 10c,

Vacuum pump 11,

Air intake 12,

Fan 13,

Input device 14,

Cooler, for example Peltier element 15,

Temperature sensor 16,

Temperature sensor 17,

Seal for filter plate 18,

Air outlet 19,

Power supply 20,

Cooling air 21,

Cooling fins for the Peltier element 22,

Cooler 23,

Baffle plate 24,

Upper part of the filtration unit 25,

Lower part of the filtration unit 26,

Air outlet for cooling air 27.

### EXAMPLES

When different normal plasmas are separated from microparticles with the aid of the described apparatus, the following results can typically be obtained:

| Sample | Microparticle | Microparticle | Percent of |
|---|---|---|---|
| number | content unfiltered | content filtered | microparticles removed % |
| 1 | 33,454 | 1,531 | 95.42% |
| 2 | 6,976 | 1,177 | 83.12% |
| 3 | 62,895 | 1,391 | 97.78% |
| 4 | 3,248 | 741 | 77.16% |
| 5 | 8075 | 1,494 | 81.49% |
| 6 | 20,202 | 4,272 | 78.85% |

Use of Microparticle-Free Plasma for Coagulation Analyses:

The following table shows different parameters, wherein under PPP the results are listed that relate to the platelet-poor plasma after centrifuging and under MPFP (micro particle filtered plasma) those that were carried out on the filtered plasma:

| Parameter | PPP | MPFP | Difference |
|---|---|---|---|
| Fibrinogen mg/dl | 285 ± 73 | 290 ± 74 | ns |
| aPTT (reagent 1) sec | 38.9 ± 2.9 | 39.5 ± 2.3 | ns |
| aPTT (reagent 2) sec | 36.9 ± 3.9 | 38.2 ± 3.4 | ns |
| aPTT (reagent 3) sec | 36.0 ± 3.5 | 38.2 ± 3.4 | p < 0.05 |
| FVIII % | 127 ± 30 | 126 ± 30 | ns |
| PT (reagent 1) % | 113 ± 19 | 114 ± 22 | ns |
| PT (reagent 2) % | 157 ± 31 | 161 ± 32 | ns |
| PT (reagent 3) % | 110 ± 11 | 113 ± 14 | ns |
| TGA peak thrombin nM | 331 ± 40 | 222 ± 26 | p < 0.05 |
| TGA AUC nM thrombin | 4163 ± 129 | 3641 ± 233 | p < 0.05 |
| Lupus LCA index | 47.4 ± 7 | 34.8 ± 6 | p < 0.05 |

These data show that the measurement of the parameters, in particular individual coagulation parameters such as aPTT, thrombin generation (TGA) and lupus tests are considerably influenced by the presence of microparticles. The deviations show that more reliable results can be achieved through the invention that are no longer dependent on the content of microparticles in the blood or the plasma.

Plasma-free microparticles produced with an apparatus according to the invention is generally well suited for use in laboratory diagnostics, in particular in coagulation diagnostics, and for use in the determination of thrombin generation.

List of references:

Jy W, Horstmann L L, Jimenez J J et al., Measuring circulating cell-derived microparticles. J. Thromb. Haemost. 2004; 2: 1842-1843.

Horstmann L L, Jy W, Jimenez J J, Bidot C, Ahn Y S. New horizons in the analysis of circulating cell-derived microparticles. Keio J Med. 2004; 53: 210-230.

The content of the two above-referenced publications is incorporated by reference in its entirety into the present specification.

1. A method for the treatment of blood plasma for the determination of one or more blood parameters, wherein the blood plasma is suctioned through a filter by a vacuum.

2. The method according to claim 1, wherein the filter has a transmission limit between 0.05 and 1.5 μm.

3. The method according to claim 1 wherein the vacuum is −50 mbar to −1000 mbar.

4. The method according to claim 1 wherein the impingement of the filter with a vacuum lasts between 30 seconds and 30 minutes.

5. An apparatus for the treatment of blood plasma for the determination of one or more blood parameters, wherein the apparatus has a filter, a collecting vessel downstream of the filter, and a vacuum pump connected to the region downstream of the filter.

6. The apparatus according to claim 5 wherein the filter has a transmission limit between 0.05 and 1.5 μm.

7. The apparatus according to claim 5 wherein the collecting vessel is a microtitration plate.

8. The apparatus according to claim 5 wherein the collecting vessels are Eppendorf tubes.

9. The apparatus according to claim 5 wherein the filter, the collection vessel and the vacuum pump are integrated into a common unit.

10. The apparatus according to claim 5 wherein a cooler is provided under the filter to cool the filtering operation.

11. The method according to claim 1 wherein the filter has a transmission limit between 0.1 and 1.2 μm.

12. The method according to claim 1 wherein the vacuum is −300 mbar to −600 mbar.

13. The apparatus according to claim 5 wherein the filter has a transmission limit between 0.1 and 1.2 μm.
